(19) United States
(12) Patent Application Publication
(10) Pub. No.: US 2012/0003686 A1
(43) Pub. Date: Jan. 5, 2012

(54) CULTURE PLATE HOLDER

(76) Inventors: Manjiri T. Kshirsagar, Woodbury, MN (US); Kurt J. Halverson, Lake Elmo, MN (US); James E. Aysta, Stillwater, MN (US)

(21) Appl. No.: 13/152,303

(22) Filed: Jun. 3, 2011

Related U.S. Application Data
(60) Provisional application No. 61/360,195, filed on Jun. 30, 2010.

(51) Int. Cl. C12M 1/22; C12M 1/00; C12Q 1/04; C12M 1/34

(52) U.S. Cl. 435/34; 435/305.1; 435/288.3; 435/307.1

(57) ABSTRACT

The present disclosure describes holder devices, testing kits that include the holder devices, and methods for using the holder devices to allow a user to dispense an aqueous test sample from a sampling device onto a culture plate device having a cover sheet, whereby the cover sheet is supported in an open position by the holder while the user dispenses the aqueous test sample onto a base member of the culture plate device.

### CROSS REFERENCE TO RELATED APPLICATION

This application claims the benefit of U.S. Provisional Patent Application No. 61/360,195 (Attorney Docket No. 66286US002), filed Jun. 30, 2010, the disclosure of which is incorporated by reference herein in its entirety.

### FIELD OF THE DISCLOSURE

The disclosure relates to holders for culture plate devices and sampling devices, and also relates to testing kits for performing sample analysis.

### BACKGROUND

Biological safety is a paramount concern in modern society. Testing for biological contamination in foods or other materials has become an important and sometimes mandatory requirement for developers and distributors of food products. Biological testing is also used to identify bacteria or other agents in laboratory samples such as blood samples taken from medical patients, environmental samples, laboratory samples developed for experimental purposes, and other types of biological samples. Various techniques and devices can be utilized to improve biological testing and to streamline and standardize the biological testing process.

A wide variety of dry culture media plates have been developed. As one example, dry culture media plates have been developed by 3M Company (hereafter “3M”) of St. Paul, Minn. Dry culture media plates are sold by 3M under the designation PETRIFILM plates. Dry culture media plates can be utilized to facilitate the rapid growth and detection of bacteria or other biological agents commonly associated with food contamination, including, for example, aerobic bacteria, E. coli, coliform, enterobacteriaceae, yeast, mold, Staphylococcus aureus, Listeria, and Salmonella. The use of PETRIFILM plates, or other growth media, can simplify bacterial testing of food samples.

Dry culture media plates can be used to enumerate or identify the presence of bacteria so that corrective measures can be performed (in the case of food testing) or proper diagnosis can be made (in the case of medical use). In other applications, dry culture media plates may be used to rapidly grow bacteria or other biological agents in laboratory samples, e.g., for experimental purposes.

Published International (PCT) Application No. U.S. 2008/069485 describes a modular system and method for detecting microorganisms, including sample loading and sample preparation.

### SUMMARY

The dispensing of a test sample onto a culture plate device may appear to be simple enough, and indeed is carried out daily around the world by users having at least one hand with an opposable thumb, using some variant of a pipette to dispense a liquid test sample onto a culture plate device. A culture plate device normally includes a cover to keep out contaminants that might interfere with a use of the culture plate device, which is typically to support the biological growth of microorganisms coming only from a test sample. There is a need to lift the cover temporarily to allow a user to dispense a test sample onto the culture plate device, and the steps of lifting the cover and holding the cover open may require the use of a hand otherwise occupied with handling a sampling device or other task, inviting various improvisations for lifting the cover and holding it open while the user dispenses the test sample. In cases where the cover is a sheet of flexible film partially attached to the culture plate device, there may be a tendency for the cover sheet to return to a closed position in the absence of some type of holding device. Manually closing the cover sheet after inoculation introduces additional variability, depending on the rate and angle in which the user returns the cover to the closed position. Rather than leaving it to the user to improvise various means of holding open the cover sheet and returning it to the closed position, and thereby risking the introduction of contaminants and variability, the current disclosure provides a holder device that supports the cover sheet and thereby aids the dispensing of test samples onto a culture device. When the culture device is removed from the holder, the cover returns to the closed position in a consistent manner. The current disclosure also describes sampling devices and concentration agents useful to include in a kit along with the holder devices, and methods for using the sampling devices in combination with the holder devices. Surprisingly, it was found that the use of a holder device of the current disclosure provided an additional advantage of reducing factors that contribute to noise and variability in the use of some culture plate devices as detection tools.

In one aspect, the present disclosure includes a holder device that allows a user to dispense a test sample from a sampling device to a culture plate, the culture plate including a culture plate base member having a sample zone and a cover sheet having a sample contact portion covering the sample zone, the holder device comprising: (a) a housing comprising a support surface arranged to support the culture plate base member; and (b) a first cover support member arranged in spaced relationship to the support surface, thereby to support a portion of the cover sheet, whereby at least a portion of the sample zone is exposed and a user is allowed to dispense a test sample into the sample zone. In some embodiments, the holder device includes first and second guide member portions that define a linear insertion path for the culture plate base member. In some embodiments of the holder device, the first and second guide member portions include first and second guide tracks, wherein each guide track has a first end and a second end, and the first ends of the guide tracks are arranged to define an insertion slot adjacent to a first end of the first cover support member.

In some embodiments, the holder device includes a stop member positioned to define an axial limit of the linear insertion path.

In some embodiments, the holder device includes a second cover support member in spaced relationship to the support surface, and the first and second cover support members are arranged to support side portions of the cover sheet. In some embodiments, the first and second cover support members each include a ramp portion arranged to support a leading edge of the cover sheet when the culture plate base member is inserted into the insertion slot. In further embodiments, the housing of the holder device includes a front wall having an opening defined therein, and wherein side portions of the front wall include the first and second cover support members, whereby a gap is formed between the housing and the sample contact portion of the cover sheet. In further embodiments of the holder device, the housing includes a top wall having an opening defined therein to allow a user to dispense a test sample from a sampling device into an interior volume of the housing. In some further embodiments of the holder device, the housing comprises a lower housing portion, an upper housing portion defining a top opening, and a shoulder portion defining an opening between the lower portion and the upper portion, the shoulder portion and upper housing portion dimensioned to support a sampling device. In some embodiments of the holder device, the upper portion includes a circumferential wall, and may further include an O-ring seated on the shoulder portion and abutting an inner surface of the circumferential outer wall.

In some embodiments of the holder device the lower housing portion is substantially rectangular and includes a front wall, a back wall, first side wall, a second side wall, and a top wall that includes the shoulder portion. In further embodiments, an observation opening is defined in any of the first side wall, the second side wall, and the back wall.

In an embodiment, the current disclose includes a holder device that allows a user to dispense a test sample from a sampling device to a culture plate, the culture plate including a culture plate base member having a sample zone and a cover sheet having a sample contact portion covering the sample zone, the holder device including:

(a) a housing, the housing including:
(i) a base portion having a support surface;
(ii) a lower housing portion extending upwards from the support surface, the lower housing portion defining a lower housing interior region and having a front wall, the front wall having an insertion slot defined therein;
(iii) an upper housing portion extending upwards from a top surface of the lower housing portion;
(iv) a shoulder portion between the upper housing portion and the lower housing portion, the shoulder portion defining a first opening;
(v) a guide member portion on an interior surface of the lower housing portion and dimensioned to support the culture plate base member, the guide member being aligned with the insertion slot and defining an insertion path; and

(b) a first cover support member arranged in spaced relation to the insertion slot; thereby to support a portion of the cover sheet, whereby at least a portion of the sample zone is exposed and a user is allowed to dispense a test sample into the sample zone. In a further embodiment, the holder device includes a second cover support member in spaced relationship to the support surface, the first and second cover support members arranged to support side portions of the cover sheet.

In another aspect, the present disclosure includes a testing kit, the testing kit including (a) a holder device of the current disclosure, and (b) at least one thin film culture plate device.

In a further embodiment the testing kit includes a sampling device, wherein the sampling device includes a first reservoir having a first opening and at least one resealable external opening; a second reservoir having a second opening;

an element comprising
i) a housing; and
ii) a movable feature residing within the housing, the movable feature having at least a first location and a second location, wherein an interior of the second reservoir is located within the movable feature and the second opening resides on the exterior of a portion of the movable feature, the first reservoir located above the element when the sampling device is in an upright position; and a second external opening, the second external opening located below the element when the sampling device is in the upright position, wherein at the first location a first passageway connects the first reservoir to the second reservoir so that the first opening is in fluid communication with the second opening, wherein at the second location a second passageway connects the second reservoir to the second external opening so that the second opening is in fluid communication with the second external opening.

In another embodiment, the kit of the current disclosure, the sampling device includes a first reservoir having a first opening, a first resealable external opening and a first volume; a second reservoir having a second opening, a second external opening and a second volume; and a plunger having a seal, the seal residing on a portion of the plunger proximate to a distal end of the plunger, the seal isolating the second volume of the second reservoir from the first volume of the first reservoir, the second volume removed through the second external opening, wherein the first reservoir is located above the second reservoir when the sampling device is in an upright position.

In some embodiments, the kit of the current disclosure has a ratio of the first volume to the second volume is in a range of about 10:1 to about 1000:1. In some embodiments, the second volume is about 1 milliliter.

In some embodiments of the above kits, a concentration agent is included wherein the concentration agent is a dispersable particulate material selected from the group consisting of particles with affinity ligands, particles without affinity ligands, antibodies or antigen binding fragments, receptors and combinations thereof. In some embodiments, the concentration agent comprises metal silicates. In some embodiments, the concentration agent comprises spheroidized magnesium silicate. In some other embodiments, the concentration agent comprises surface treated diatomaceous earth, the surface treatment selected from the group consisting of titanium dioxide, nanoscale gold, nanoscale platinum and combinations thereof. In some other embodiments, the concentration agent comprises gamma-FeO(OH).

In some embodiments, kits of the current disclosure may further include a detection agent and a growth medium.

In some embodiments, kits of the current disclosure may further include a plurality of holder devices.

In another aspect, the current disclosure includes a method that allows a user to dispense an aqueous test sample from a sampling device to a thin film culture plate device, the method comprising steps of:

(a) providing a holder device of any one of claims 8 to 15;

(b) providing a test sample contained in a sampling device having a second reservoir and a second external opening;

(c) providing a culture plate device including a plate base member having a sample zone and a cover sheet having a sample contact portion covering the sample zone

(d) abutting a portion of the cover sheet against the first cover support member;

(e) inserting a leading edge of the plate base member into the insertion slot and urging the plate base member along the insertion path;

(f) inserting the second external opening of the sampling device into an opening in the housing; and

(g) dispensing the aqueous test sample from the second reservoir onto the plate base member.

In some embodiments of the method of the current disclosure, the cover sheet has a tab portion extending beyond the leading edge of the plate base member, and abutting the tab portion against the first cover support member thereby to lift a portion of the cover sheet from the culture plate base member.

In some embodiments, step (e) of the method further includes visually confirming a positioning of the thin film culture plate device within the lower housing portion.

In some embodiments, the method of the current disclosure includes after step (g) a step of sliding the culture plate device back along the insertion path and out of the holder device.

In some embodiments, the method of the current disclosure may be repeated with another culture plate device. In other embodiments, the method of the current disclosure may be carried out using an automation system to position the culture plate device and/or the sampling device.

As included in this specification and the appended claims, the singular forms “a”, “an”, and “the” include plural referents unless the content clearly dictates otherwise. Thus, for example, reference to a composition containing “a compound” includes a mixture of two or more compounds. As used in this specification and appended claims, the term “or” is generally employed in its sense including “and/or” unless the content clearly dictates otherwise. Also herein, the recitation of numerical ranges by endpoints includes all numbers subsumed within that range (e.g., 1 to 5 includes 1, 1.5, 2, 2.75, 3, 3.8, 4, and 5).

Unless otherwise indicated, all numbers expressing quantities or ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present disclosure. At the very least, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains errors necessarily resulting from the standard deviations found in their respective testing measurements.

### GLOSSARY

As used in this patent application:
“detection” means the identification of at least a component of a microorganism, which thereby determines that the microorganism is present;
“aqueous test sample” means a liquid sample to be analyzed that includes water and may also include a microorganism-bound concentration agent, which may be present as particulates in the water;
“microorganism” means any cell having genetic material suitable for analysis or detection (including, for example, enveloped viruses);
“microorganism-bound concentration agent” means a composition comprising a microorganism bound to or captured by a concentration agent.

### BRIEF DESCRIPTION OF THE DRAWINGS

FIG. 1A is a perspective view of a culture plate device; FIG. 1B is a top view of a culture plate device;

FIG. 2 is a perspective view of a holder device of the current disclosure;

FIG. 3 is a cross-sectional view of a holder device of the current disclosure, taken generally along line 3-3, FIG. 2;

FIG. 4A is a perspective view of a kit that includes a sampling device, a holder device, and a culture plate device, and FIG. 4B shows these components of the kit being used in combination as a system for dispensing;

FIGS. 5A, 5B, and 5C are partially cutaway views of a sampling device having a first reservoir, a second reservoir, and a plunger in various positions; the plunger in FIG. 5A is positioned above the second reservoir; the plunger in FIG. 5B is positioned to seal the second reservoir; the plunger in FIG. 5C is positioned extending through the second reservoir;

FIGS. 6A and 6B are partially cutaway views of a sampling device having a first reservoir, a second reservoir, and an element including a movable feature; the movable feature in FIG. 6A is in a sample collecting position; the movable feature in FIG. 6B is in a sample dispensing position;

FIG. 7 is a side view of the element of FIGS. 6A and 6B that includes a movable feature;

FIG. 8A is a perspective view of an embodiment of a holder device of the current disclosure; FIG. 8B is a cross-sectional view of the holder in FIG. 8A, taken generally along line 8B-8B, FIG. 8A;

FIG. 9A is a perspective view of an embodiment of a holder device of the current disclosure; FIG. 9B is a cross-sectional view taken generally along line 9B-9B, FIG. 9A;

FIG. 10A is a perspective view of an embodiment of a holder device of the current disclosure; FIG. 10B is an enlarged side view of the area indicated in FIG. 10A.

FIGS. 11A and 11B are partially cutaway views of a sampling device positioned on a holder device of the current disclosure (shown in cross-sectional view) and dispensing a test sample to the plate base member of a culture plate device (shown in cross-sectional view).

Like reference numbers in the various figures indicate like elements. Some elements may be present in identical or equivalent multiples; in such cases only one or more representative elements may be designated by a reference number but it will be understood that such reference numbers apply to all such identical elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated. Although terms such as “top”, bottom”, “upper”, lower”, “under”, “over”, “front”, “back”, “outward”, “inward”, “up” and “down”, and “first” and “second” may be used in this disclosure, it should be understood that those terms are used in their relative sense only unless otherwise noted. In particular, in some embodiments certain components may be present in interchangeable and/or identical multiples (e.g., pairs). For these components, the designation of “first” and “second” may apply to the order of use, as noted herein (with it being irrelevant as to which one of the components is selected to be used first).

### DETAILED DESCRIPTION

The present disclosure describes holder devices, testing kits that include the holder devices, and methods for using the holder devices to allow a user to dispense an aqueous test sample from a sampling device onto a culture plate device having a cover sheet, whereby the cover sheet is supported in an open position by the holder while the user dispenses the aqueous test sample onto a base member of the culture plate device. In some embodiments, holder devices of the present disclosure may be used to facilitate dispensing a concentrated aqueous test sample from a sampling device onto a culture plate device. Suitable sampling devices, concentration agents, and methods for concentrating a large volume sample having a low concentration of microorganisms are described in International (PCT) Application No. U.S. 2009/069780, International (PCT) Application No. U.S. 2008/078575, and International (PCT) Application No. U.S. 2008/078587, the disclosures of each of which are incorporated herein by reference for this purpose.

### Culture Plate Devices

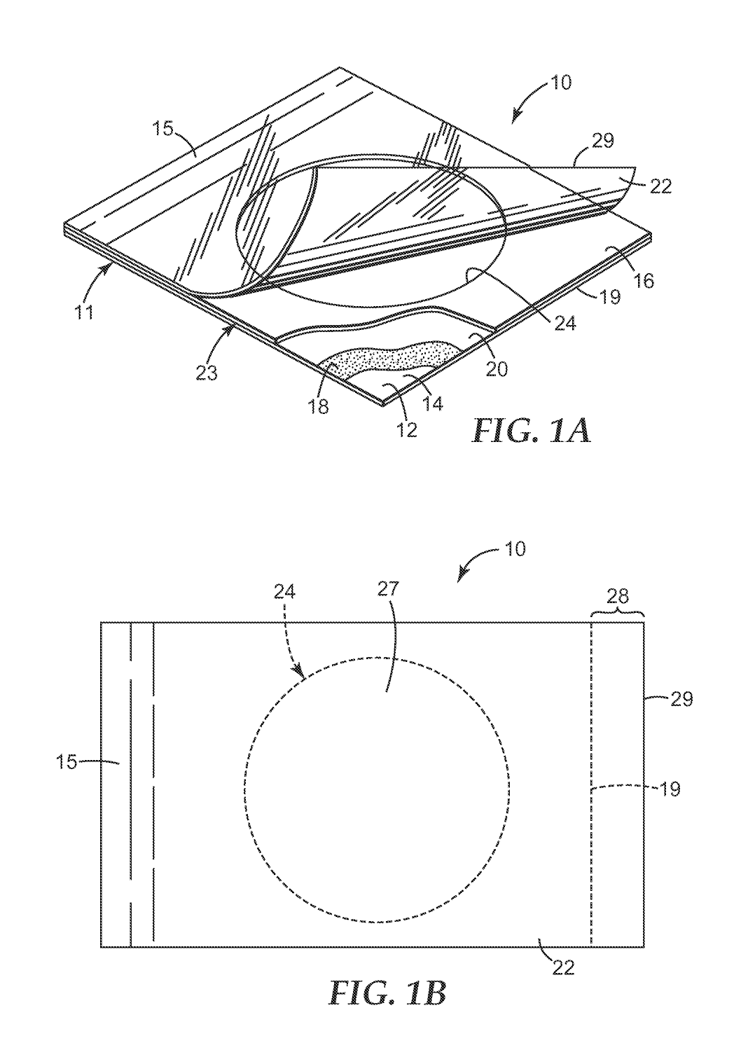

In some embodiments, the current disclosure includes culture plate devices, which may include thin film culture plate devices. Thin film culture plate devices are typically more compact than traditional agar Petri dishes and typically contain dry, rehydratable culture medium to support the growth of certain microorganisms. FIG. 1A illustrates an embodiment of a culture plate device 10 including plate base member 11 comprising several layers, including a self-supporting substrate layer 12 and a spacer layer 23. Substrate layer 12 is coated on its upper surface 14 with a layer of adhesive composition 18. Cold-water-soluble powder, comprising one or more gelling agents, is adhered in a thin, relatively uniform layer 20 to the adhesive composition 18. Once inoculated with an aqueous test sample (not shown), the layer of cold-water-soluble powder 20 quickly hydrates to form a reconstituted medium (not shown), which in turn is capable of growing microorganisms present in an aqueous inoculum. Spacer layer 23 partially covers substrate 12 and the surface of powder 20 and contains aperture 24. It will be understood that the area within aperture 24 in this embodiment is a “sample zone,” that is, the area on a culture plate device where a test sample is dispensed. Culture plate device 10 also includes cover sheet 22, to cover the reconstituted medium formed after addition of the aqueous test sample. As shown in FIG. 1B, cover sheet 22 includes a sample contact portion 27, which overlays the sample zone within aperture 24. In the embodiment shown in FIG. 1B, cover sheet 22 is slightly longer than plate base member 11, and includes a cover sheet leading edge 29 and a tab portion 28 overlaying base member leading edge 19. In some other embodiments, cover sheet 22 may be coextensive with plate base member 11. A benefit of having a tab portion 28 may include facilitating the lifting of cover sheet 22 by hand, or by abutting cover sheet leading edge 29 against a cover lift member on a holder device of the current disclosure (described below).

In some embodiments, cover sheet 22 is attached to a portion of plate base member 11 by a hinge 15, which in some embodiments may include an adhesive layer between a portion of cover sheet 22 and a portion of upper surface 16 of plate base member 11. Hinge 15 is not limited to including an adhesive, and other types of hinges are suitable, for example a hinge including laminated materials.

In a closed position, cover sheet 22 covers upper surface 16 of plate base member 11, and in an open position, cover layer 22 is at least partially lifted up from upper surface 16 of plate base member 11 thereby exposing a portion of the sample zone.

Additional non-limiting descriptions of culture plate devices and methods of using are disclosed in U.S. Pat. No. 4,565,783, U.S. Pat. No. 5,089,413, and U.S. Pat. No. 5,681,712, the disclosures of each of which is incorporated herein by reference.

### Holder Devices

Holder devices of the current disclosure each include features for supporting a culture plate device base member and supporting a cover sheet, and in some embodiments the holder devices include features for holding a sampling device.

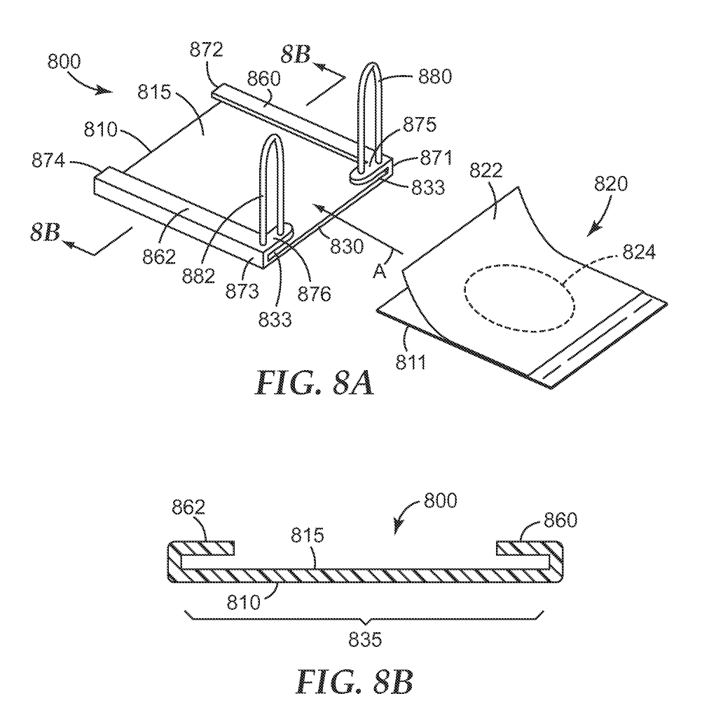

Beginning with the simpler embodiments, FIGS. 8A and 8B are views of an embodiment of a holder device 800 that includes a housing 810 having a support surface 815 arranged to support a culture plate base member 811 of culture plate device 820, wherein the support surface includes a first guide member portion 860 and second guide member portion 862 that together define a linear insertion path for culture plate base member 811. First guide member portion 860 includes a first guide track having a first end 871 and a second end 872, and second guide member portion 862 includes a second guide track having a first end 873 and a second end 874, and first ends 871 and 873 define an insertion slot 833. FIG. 8B is a sectional view taken general along line 8B-8B, FIG. 8A, showing first guide portion 860 and second guide portion 862 defining a cross-path width 835, dimensioned to permit insertion of plate base member 811.

Referring again to FIG. 8A, the illustrated embodiment includes a first cover support member 880 arranged in spaced relationship to support surface 815, thereby to support a portion of a cover sheet 822 of culture plate device 820, whereby at least a portion of sample zone 824 on is exposed and a user is allowed to dispense a test sample into sample zone 824. As shown in FIG. 8A, holder device 800 includes a second cover support member 882 in spaced relationship to the support surface, the first and second cover support members 880 and 882 are arranged to support side portions of cover sheet 822, leaving a gap between holder device 800 and the sample contact portion of cover sheet 822. It will be understood that in some embodiments, cover support members 880 and 882 may comprise a rounded surface to minimize contact between any portion of cover sheet 822 and holder device 800, thereby to minimize opportunity for contamination of any portion of the cover sheet.

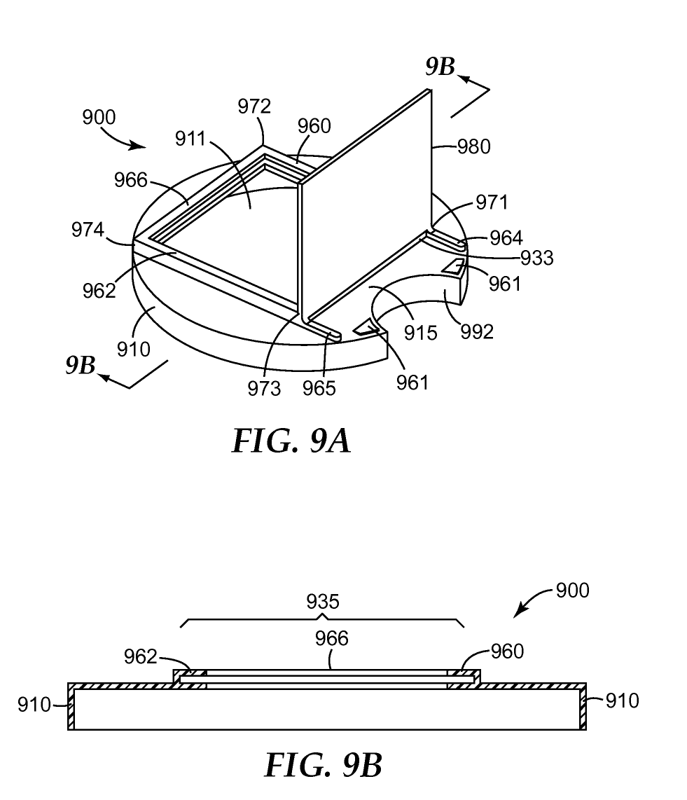

FIG. 9A schematically illustrates an embodiment of a holder device 900 that includes a housing 910 having a support surface 915 arranged to support a culture plate base member 11 of culture plate device 20 (from FIG. 1), wherein the support surface includes a first guide member portion 960 and second guide member portion 962 that together define a linear insertion path for culture plate base member 11. First guide member portion 960 includes a first guide track having a first end 971 and a second end 972, and second guide member portion 962 includes a second guide track having a first end 973 and a second end 974, and first ends 971 and 973 define an insertion slot 933. Guide fingers 964 and 965 are optionally provided to aid in directing culture plate base member 11 into insertion slot 933, and extra support portions 961 are optionally provided on support surface 915 to aid with keeping culture plate base member 11 in an inserted position along the linear insertion path.

Holder device 900 also includes a stop member 966, which in the embodiment shown in FIG. 9A is positioned across the second ends 972 and 974 of guide member portions 960 and 962. Also shown in FIG. 9A is an opening 911 defined in support surface 915, which may permit a user to direct light through either major surface of the culture plate device when inserted into the holder device and thereby enhance viewing.

FIG. 9B is a sectional view taken general along line 9B-9B, FIG. 9A, showing first guide portion 960 and second guide portion 962 defining a cross-path width 935, dimensioned to permit insertion of a plate base member 11.

Referring again to FIG. 9A, the illustrated embodiment includes a first cover support member 980 arranged in spaced relationship to support surface 915, thereby to support a portion of a cover sheet 22 of culture plate device 20 (from FIG. 1), whereby at least a portion of sample zone 24 on is exposed and a user is allowed to dispense a test sample into sample zone 24. In the embodiment shown in FIG. 9A, first cover support member 980 may contact the sample contact portion 27 of cover sheet 22. While it is generally desirable to avoid contacting sample contact portion 27 against a holder device, there may be select instances where, for example, sufficient antibiotic or other selective agents are present in the culture plate growth media so that it is not consequential for sample contact portion 27 to come into contact with a cover support member.

In an alternative embodiment of a use of a holder device 900, insertion slot 133 may be dimensioned to accommodate the insertion of a culture plate device 10 after inoculation and incubation, and leaving cover sheet 22 in contact with plate base member 11 (i.e., not abutting cover sheet 22 against any cover support member). A user may then direct light through opening 911 and visually inspect culture plate device 10 for growth of microorganisms, or an automated reader device may be used for collecting and analyzing an image of the sample zone. In this alternative use, a cover support member may be present, but its presence is not required for this post-incubation handling of culture plate device 10.

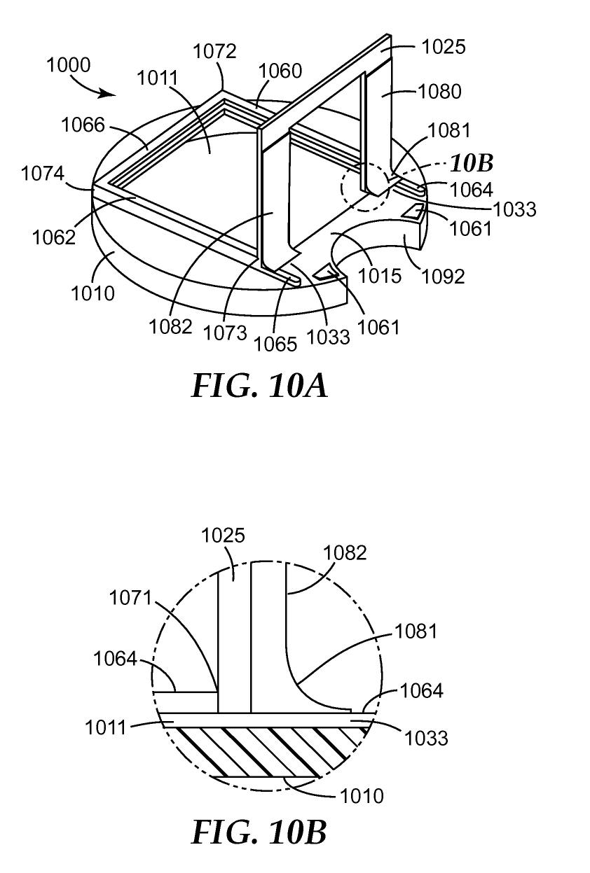

FIG. 10A schematically illustrates an embodiment of a holder device 1000 that includes a housing 1010 having a support surface 1015 arranged to support a culture plate base member 11 of culture plate device 20 (from FIG. 1A), wherein the support surface includes a first guide member portion 1060 and second guide member portion 1062 that together define a linear insertion path for culture plate base member 11. First guide member portion 1060 includes a first guide track having a first end 1071 and a second end 1072, and second guide member portion 1062 includes a second guide track having a first end 1073 and a second end 1074, and first ends 1071 and 1073 define an insertion slot 1033. Guide fingers 1064 and 1065 are optionally provided to aid in directing culture plate base member 11 into insertion slot 1033, and extra support portions 1061 are optionally provided on support surface 1015 to aid with keeping culture plate base member 11 in an inserted position along the linear insertion path.

Holder device 1000 also includes a stop member 1066, which in the embodiment shown in FIG. 10A is positioned across the second ends 1072 and 1074 of guide member portions 1060 and 1062. Also shown in FIG. 10A is an opening 1011 defined in support surface 1015, which may permit a user to direct light through either major surface of the culture plate device when inserted into the holder device and thereby enhance viewing.

The illustrated embodiment in FIG. 10A includes a first cover support member 1080 arranged in spaced relationship to support surface 1015, thereby to support a portion of a cover sheet 1022 of culture plate device 1020, whereby at least a portion of sample zone 24 (refer to FIG. 1A) on is exposed and a user is allowed to dispense a test sample into sample zone 24. As shown in FIG. 10A, holder device 1000 includes a second cover support member 1082 in spaced relationship to the support surface, the first and second cover support members 1080 and 1082 are arranged to support side portions of cover sheet 22, leaving a gap between holder device 1000 and the sample contact portion of cover sheet 22.

First cover support member 1080 includes a ramp portion 1081 arranged to support a leading edge 29 of cover sheet 22. FIG. 10B is an enlarged illustration showing ramp portion 1081 located on cover support member 1080 and adjacent to insertion slot 1033. As shown in FIG. 10A, cover support member 1082 has a ramp portion similar to 1081. The optional ramp portions on cover support members 1080 and 1082 may be useful in catching leading edge 29 of cover sheet 22 when plate base member 11 is inserted into the insertion slot.

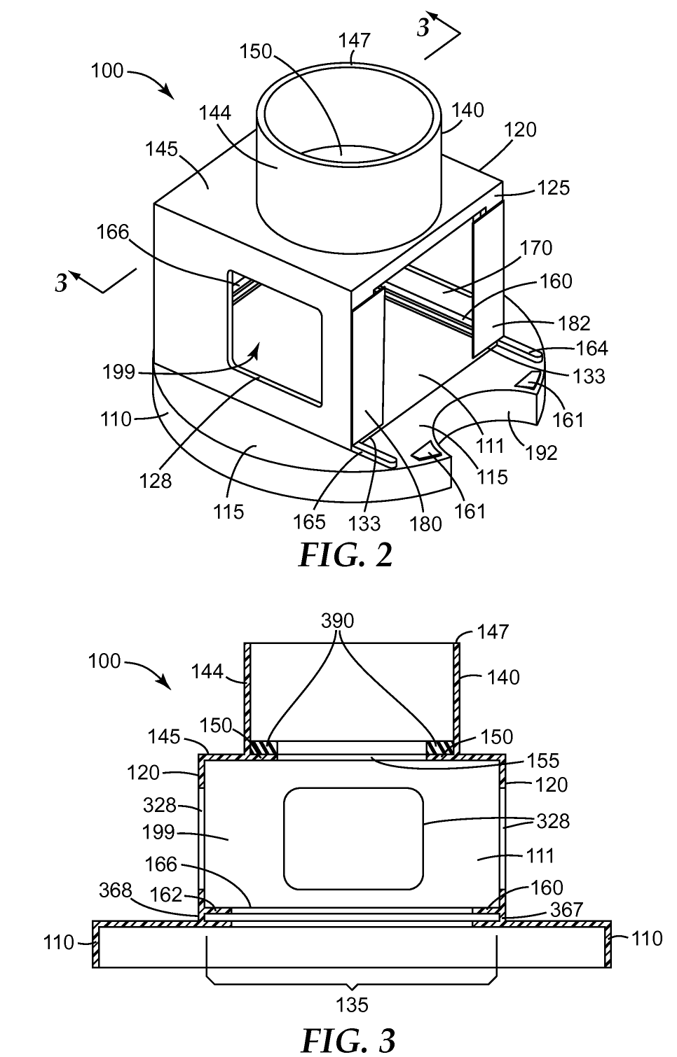

In some other embodiments of a holder device, provision is made for holding a sampling device in a position suitable to allow a user to dispense a test sample from the sampling device onto the sample zone of a culture plate device 10 (refer to FIG. 1A for an illustration of culture plate device 10). Shown in FIG. 2 is a schematic perspective view of an embodiment of a holder device 100 of the current disclosure, wherein holder device 100 allows a user to dispense a liquid sample to a culture plate device 10 from a sampling device. Holder device 100 is shown in an upright position and includes a base portion 110 having a support surface 115. Lower housing portion 120 extends outwardly from support surface 115, defining an interior portion 199. In the embodiment shown in FIG. 2, lower housing portion 120 includes a front wall 125, a top surface 145, and side portions of front wall 125 have a lower edge to define an upper edge of insertion slot 133. In some embodiments, a lower edge of insertion slot 133 may be defined by a portion of base portion 110. In the embodiment shown in FIG. 2, an upper housing 140 extends outwardly from the lower housing portion's top surface 145, and upper housing portion 140 includes a circumferential outer wall 144 that defines an annular shoulder portion 150 between upper housing portion 140 and lower housing portion 120. In the embodiment shown, circumferential outer wall 144 also defines a rim 147 at an upper edge thereof. In other embodiments, upper housing portion 140 may have any suitable shape, although in general the shape will be selected to permit a user to dispense a sample from a sampling device.

As shown in the embodiment illustrated in FIG. 2, lower housing portion 120 of holder device 100 may be substantially rectangular, including a first side wall 170, a second side wall 172, a back wall 176, and a top wall 178 that includes top surface 145 of the lower housing portion. In other embodiments, lower housing portion 120 may have any suitable shape, including, for example, a dome-shape.

In some embodiments, lower housing portion 120 of holder device 100 may include at least one observation opening 328 in any of a first side wall 170, a second side wall 172, or a back wall 176.

In the embodiment shown in FIG. 2, base 110 defines a cutaway feature 192 to accommodate a user's fingers during the insertion and/or removal of a culture plate device 10 into/from holder device 100. Cutaway feature 192 is provided as an optional convenience.

In some embodiments of holder device 100, base 110 may define an opening 111 in a central portion thereof, and typically located beneath upper housing portion 140, to permit illumination of plate base member 11 from below. As mentioned for holder device 900 above, an alternate use of the holder device may include post-incubation viewing of an inoculated sample zone in a culture plate device 10, provided that insertion slot 133 is dimensioned to accommodate insertion of a culture plate 10 having cover sheet 22 in a closed position.

As illustrated in FIG. 3, which is a sectional view taken generally along line 3-3 of FIG. 2, shoulder portion 150 defines a first opening 155 between upper housing portion 140 and lower housing portion 120. In the embodiment illustrated in FIG. 3, an O-ring 390 is seated on shoulder portion 150 and abutting an inner surface of circumferential outer wall 144.

In the embodiment shown in FIG. 3, lower housing portion 140 includes a first guide member portion 160 and a second guide member portion 162 defining a cross-path width sufficient to accommodate the insertion of a plate base member 11 (refer to FIG. 1). First and second guide member portions 160 and 162 are aligned with insertion slot 133 and may extend horizontally from a first end adjacent insertion slot 133 towards a second end adjacent back region 199 of lower housing portion 120. In some other embodiments, first and second guide member portions 160 and 162 may be portions of a unitary guide member portion, or a pair of guide tracks 367 and 368 may be portions of a unitary lower housing portion 120, or a pair of guide tracks 367 and 368 may be portions of a unitary holder device 100. A suitable guide member portion of the current disclosure may have a “C-shaped” profile of the type shown in FIG. 3 (see guide member portions 160 and 162, shown in end-on profile), although a variety of other suitable profiles may be used, including an “L-shaped” profile, for example.

In the illustrated embodiment, holder device 100 includes guide fingers 164 and 165 (FIG. 2), positioned to provide aid in directing plate base member 11 into insertion slot 133. Extra support portions 161 are optionally provided on support surface 115 to aid with keeping culture plate base member 11 in an inserted position along the linear insertion path.

Holder device 100 includes a stop member 166 positioned in spaced relation to insertion slot 133, typically located towards back region 199, and defines an axial limit of the linear insertion pathway. In the embodiment shown in FIG. 2, stop member 166 resembles the guide members 160 and 162, and is connected across first guide track 160 and second guide track 162 and thereby defines an axial limit of the insertion pathway. In other embodiments, a suitable stop member 166 may include, for example, a portion of a rear wall of lower housing portion 120.

In an embodiment, holder device 100 includes a first cover support member 180 arranged in spaced relation to insertion slot 133. In some embodiments, the first cover support member may be a portion of front wall 125. In some other embodiments, a first cover support member 180 may be a separate piece of a material adhered to a portion of front wall 125. In the embodiment shown in FIG. 2, holder device 100 includes a second cover support member 182 arranged in spaced relationship to horizontal slot 133. Cover support members of the current disclosure may include various shapes beyond those illustrated, including for example protrusions from portions of front wall 125. Each cover support member may include a ramp portion similar to the ramp portion 1081 shown in FIG. 10A.

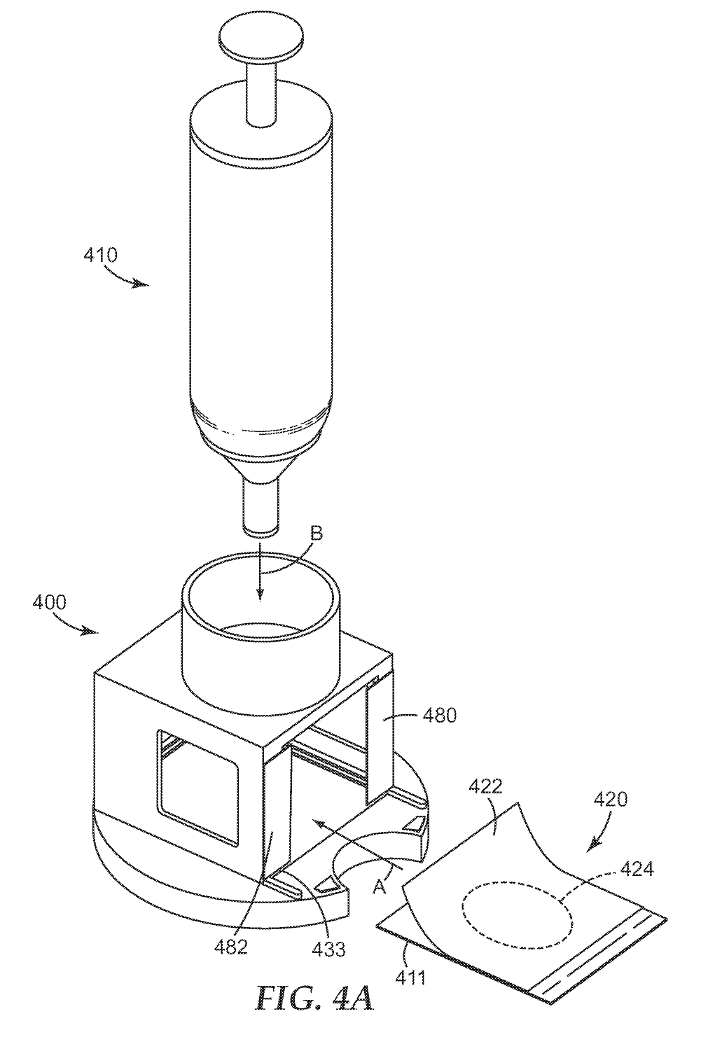

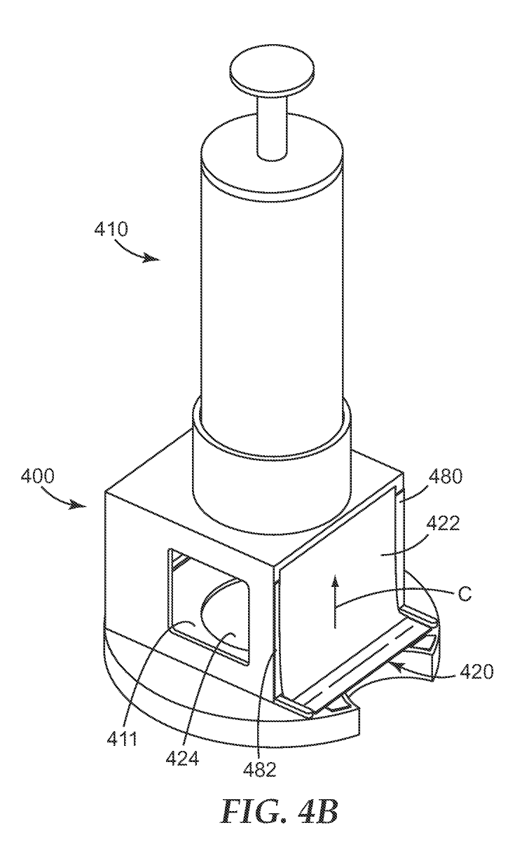

In the embodiments shown in FIGS. 4A and 4B, first and second cover support members 480 and 482 support side portions of cover sheet 422 of culture plate device 420 when plate base member 411 is inserted into holder device 400 in the direction indicated by “A”. As plate base member 411 is urged into insertion slot 433, cover sheet 422 abuts first and second cover support member 480 and 482, and the leading edge of cover sheet 422 travels in the direction indicated by arrow “C”. Cover support members of the current disclosure may include various shapes beyond those illustrated, including for example protrusions from portions of front wall 125.

Referring to FIGS. 4A and 4B, it may be desirable to keep a central portion of cover sheet 422 from contacting holder device 100, in order to avoid contamination of cover sheet 422 and thereby minimize potential for contamination of the liquid sample. It will be noted that a thin film culture plate device 420 may include a circular sample zone 424, a portion of which may be seen in FIG. 4B, circular sample zone 424 typically being about 50 millimeters in diameter and laterally centered in thin film culture plate device 420. FIG. 4A illustrates an embodiment where first cover support member 480 and second cover support member 482 are spaced apart from each other. In a specific embodiment, first cover support member 480 and second cover support member 482 are spaced apart from each other by a suitable spacing, for example about 65 millimeters, in order to minimize a potential for contacting a sample contact portion of cover sheet 422.

In some embodiments of the current disclosure, a dispensing step is facilitated by inserting a sampling device into a holder device as shown in FIGS. 4A and 4B, where sample device 410 is inserted along path “B” into holder device 400.

Holder devices of the current disclosure may be constructed from any suitable materials, including but not limited to plastic, epoxy resins, cardboard, metal, glass, or even stone. A magnet, clip, double-sided tape, or other means of attachment may be included on a bottom surface of the housing to anchor the holder device during use.

Methods for constructing holder devices of the current description include those commonly known in the art, including, for example, injection molding, stereolithography,

### Sampling Devices

In some embodiments, sampling devices described herein provide a first reservoir and a second reservoir within a sampling device. The first reservoir is designed to accommodate a large volume sample having sufficient volume for mixing of the sample and a concentration agent to form a microorganism bound concentration agent. Microorganism bound concentration agents can be, for example, dispersible in the sample, and then collected or transferred from the first reservoir to the second reservoir of the sampling device, which in some embodiments includes allowing microorganism bound concentration agent particles to settle into the second reservoir, by gravity. The second reservoir has a smaller volume than the first reservoir, in order to contain the microorganism bound concentration agent, and the smaller volume from the second reservoir is subsequently dispensed from the sampling device onto a culture card device.

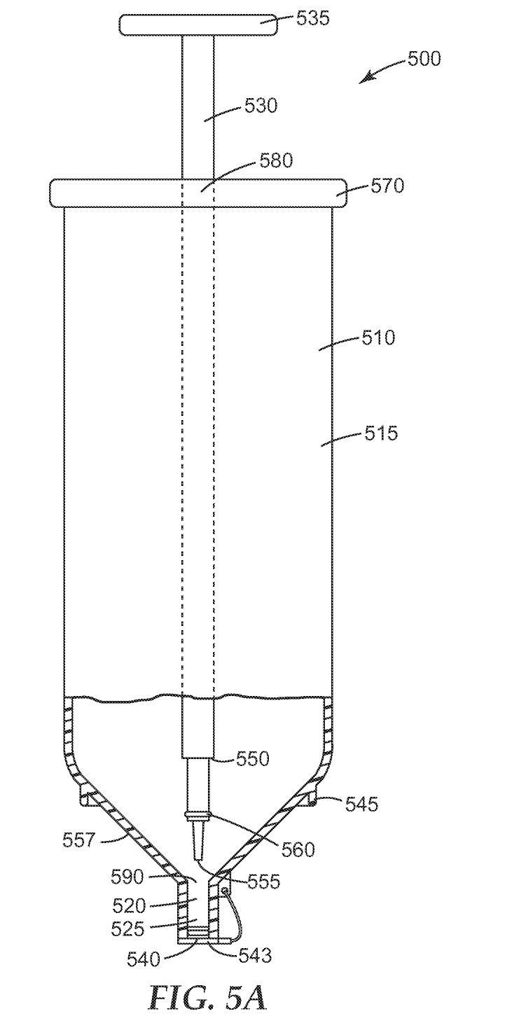

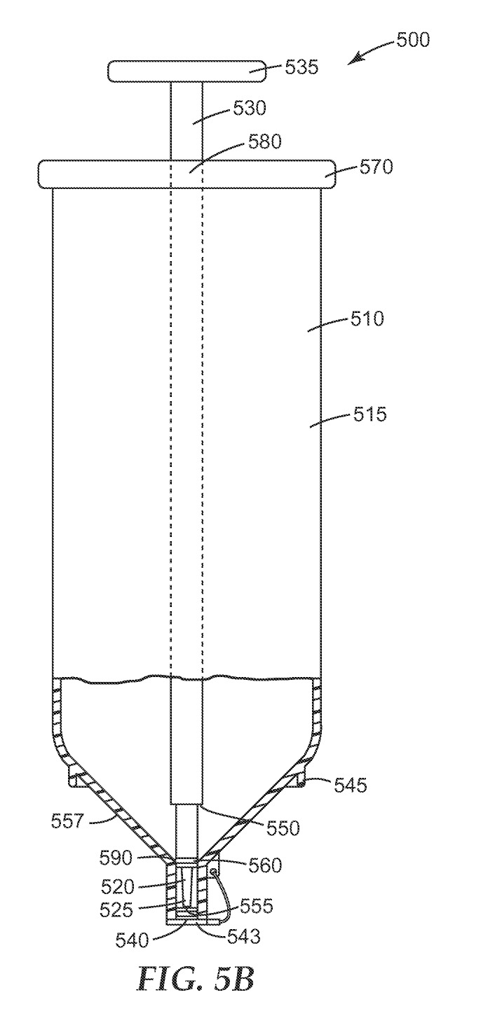

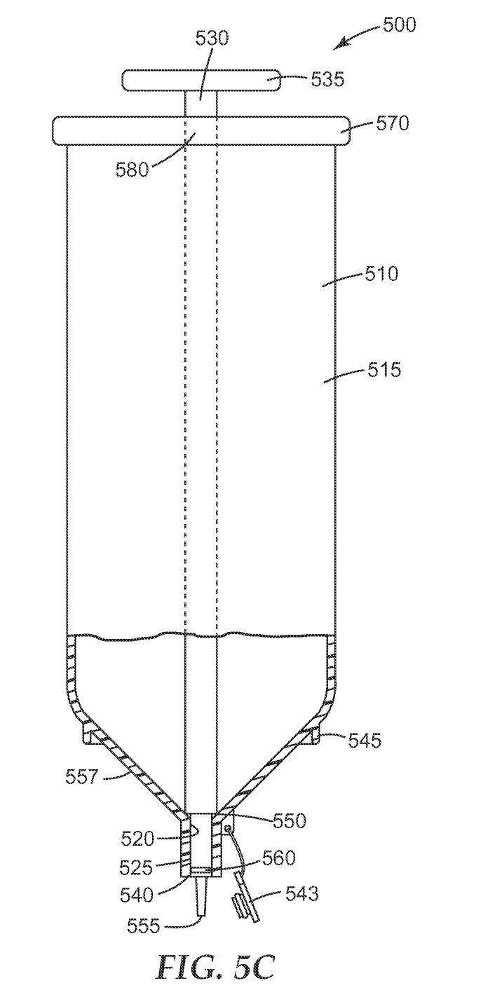

FIGS. 5A-5C illustrate an embodiment of a sampling device 500, shown in an upright position, having a first reservoir 510 located above a second reservoir 520, and also having a moveable plunger 530 shown in various positions. Sampling device 500 includes a first reservoir 510, a second reservoir 520, and a plunger 530. First reservoir 510 defines a first opening 590, a first resealable external opening 570, and a first volume 515. Second reservoir 520 defines a second resealable external opening 540, and a second volume 525. Plunger 530 has knob 535 at an upper end and extends downward through first opening 590 at an intersection 580 to a lower end 555. Plunger 530 also has a seal 560 residing on a portion of the plunger 530 proximate to lower end 555, and a plunger shoulder 550 located above seal 560. As shown in FIG. 5B, seal 560 isolates second volume 525 of second reservoir 520 from first volume 515 of first reservoir 510. FIG. 5C shows that lower end 555 of plunger 530 can be moved to extend below the second reservoir 520 and the second external opening 540, by urging cap 543 open and thereby dispensing second volume 525 from second reservoir 520 through second external opening 540. Plunger shoulder 550 may conveniently serve a stop for plunger 530 by contacting the wall of first reservoir 510 near first opening 590.

In one aspect, sampling device 500 may be utilized with a holder of the current disclosure to hold sampling device 500 in an upright position. In some embodiments, a holder device of the current disclosure may be used to hold the sampling device 500 in a stationary position.

In some embodiments of sampling device 500, a ratio of the first volume 515 to the second volume 525 may be in a range of about 10:1 to about 1000:1. In some embodiments, the first volume may be about 250 milliliters. In some embodiments, the second volume 525 may be about 1 milliliter.

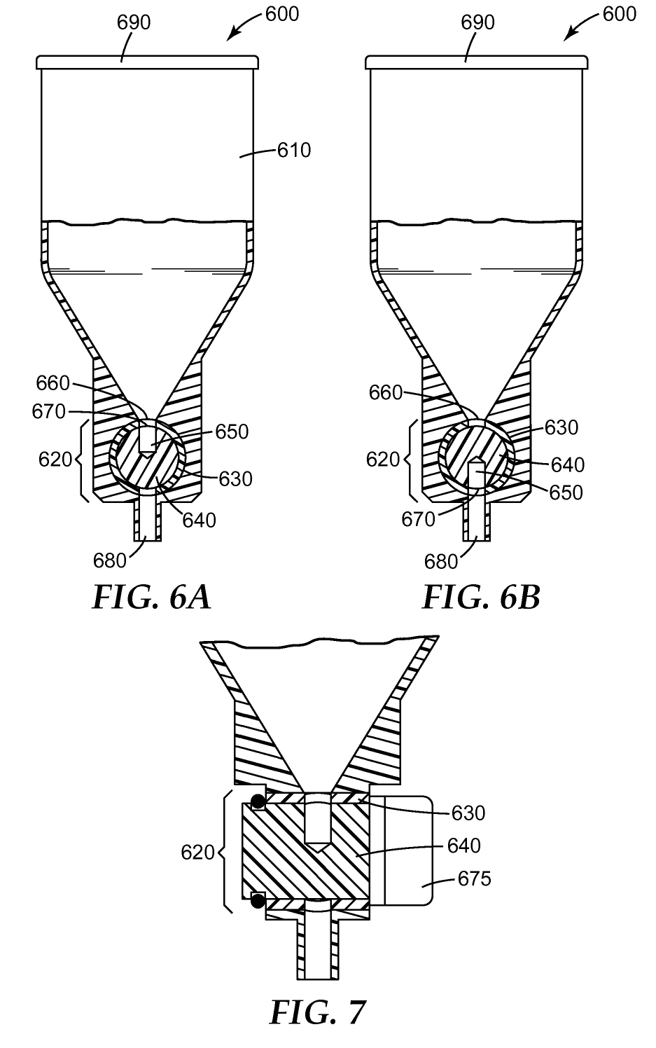

FIGS. 6A-6B schematically illustrate another embodiment of a sampling device 600 having a movable feature 640 that can be rotated to various positions. Sampling device 600 comprises: (a) a first reservoir 610 that includes a first opening 660 and at least one re-sealable external opening 690; (b) a second reservoir 650 that includes a second opening 670; (c) an element 620 that includes a housing 630 and movable feature 640 residing within housing 630; and (d) a second external opening 680. It will be understood that first reservoir 610 is located above element 620 when the sampling device 600 is in an upright position.

Movable feature 640 can be rotated to a sample collecting position as shown in FIG. 6A, and to a sample dispensing position as shown in FIG. 6B. As shown in FIG. 6A, when movable feature 640 is in the sample collecting position, first opening 660 faces second opening 670 such that first reservoir 610 is in fluid communication with second reservoir 650, thereby enabling a microorganism bound composition to settle from first reservoir 610 into second reservoir 650 via a first passageway. Second reservoir 650 typically has a fixed volume to collect the microorganism bound composition. When movable feature 640 is rotated to the sample dispensing position, shown in FIG. 6B, second opening 670 faces second external opening 680, thereby enabling the microorganism bound composition to fall from second reservoir 650 and out through second opening 680 via a second passageway.

FIG. 7 illustrates a side view of element 620. Element 620 has a housing 630 and a movable feature 640. Movable feature 640 has a first component 675 for moving the second reservoir from a first location to a second location (not shown). In some embodiments, the first component 675 protrudes from the movable feature 640 for accessibility to the movable feature 640. In embodiments where first component 675 protrudes from movable feature 640, upper housing portion 140 (see FIG. 2) may need to define a complementary cutaway (not shown) to accommodate protruding first component 675.

The sampling devices illustrated in FIGS. 5A-5C, 6A-6B and 7 can be formed from a number of materials. Materials useful for forming the sampling devices can include, for example, glass, polymeric materials, composite materials, and the like. These devices can also be constructed of more than one material. Some examples of polymeric materials include polypropylene, polycarbonate, acrylics, polystyrene, polyolefin, high density polyethylene, high density polypropylene, and the like. In some embodiments, devices can be formed from one or more methods of fabrication including, for example, injection molding, blow molding, and by other fabrication techniques.

Additional descriptions of sampling devices and methods for concentrating microorganisms are described in International (PCT) Application No. U.S. 2009/069780, the disclosure of which is incorporated herein by reference for this purpose.

### Concentration Agents

Concentration agents suitable for mixing with samples, comprising a microorganism, for providing microorganism bound compositions are described. Concentration agents are generally particulate or dispersible in the sample, and also concentrate microorganisms present in large volume samples. Such concentration agents have been found effective for capturing microorganisms. The term “concentration agent” generally refers to a material for concentrating a general population of microorganisms present in a sample. Examples of concentration agents have been described in U.S. Provisional Patent Application Ser. No. 61/141,813 and International (PCT) Published Patent Application Nos. U.S. Patent Application Nos. U.S. 2008/060574; U.S. 2008/078587; U.S. 2008/078413; U.S. 2008/078563; U.S. 2007/069460; and U.S. 2008/078575, each of which is incorporated herein by reference in its entirety.

Concentration or capture using concentration agents (e.g., capture agents), in some embodiments, can be selected to be nonspecific or specific to any particular strain, species, or type of microorganism and therefore provide for the concentration of a general population of microorganisms in a sample. In some embodiments, specific strains of microorganisms can be detected from among the captured microorganism population using any known detection method with strain-specific probes or with strain-selective culture media. Thus, the concentration agents can be used, for example, in the detection of microbial contaminants or pathogens (particularly water-borne and food-borne pathogens such as bacteria) in clinical, food, environmental, or other samples.

In carrying out the method of the present disclosure, the concentration agents can be used in any form that is amenable to sample contact and microorganism capture (for example, in particulate form or applied to a support such as a dipstick, film, filter, tube, well, plate, beads, membrane, or channel of a microfluidic device, or the like). Preferably, the concentration agents are used in particulate form, more preferably comprising microparticles (preferably, microparticles having a particle size in the range of about 1 micrometer (more preferably, about 2 micrometers) to about 100 micrometers (more preferably, about 50 micrometers; even more preferably, about 25 micrometers; most preferably, about 15 micrometers; where any lower limit can be paired with any upper limit of the range).

Concentration agents useful for carrying out the method of the present disclosure include particulate concentration agents that comprise metal, metal oxide microparticles, metal silicates, diatomaceous earth, surface modified diatomaceous earth, particles having functional groups, biomolecules, fragments of biomolecules, nanoparticles, and combinations thereof.

Some examples of concentration agents include iron, silica, titania, zirconia and others useful for collecting and concentrating samples. In some embodiments, gamma-FeO(OH) (also known as lepidocrocite) can be used as a concentration agent. Such concentration agents have been found to be more effective than other iron-containing concentration agents in capturing gram-negative bacteria, which are the microorganisms of greatest concern in regard to food- and water-borne illnesses and human bacterial infections. The concentration agents can further include (in addition to gamma-FeO(OH)) other components (for example, boehmite (α-AlO(OH)), clays, iron oxides, and silicon oxides), but, preferably, such other components do not significantly interfere with the intimate contact of the sample and the concentration agent when carrying out the method of the present disclosure. Gamma-FeO(OH) is also commercially available (for example, from Alfa Aesar, A Johnson Matthey Company, Ward Hill, Mass., and from Sigma-Aldrich Corporation, St. Louis, Mo.).

In carrying out the method of the present disclosure where the concentration agent is gamma-FeO(OH), the concentration agents can be used in particulate form, more preferably comprising microparticles (preferably, microparticles having particle sizes (largest dimension) in the range of about 3 micrometers (more preferably, about 5 micrometer; most preferably, about 10 micrometers) to about 100 micrometers (more preferably, about 80 micrometers; even more preferably, about 50 micrometers; most preferably, about 35 micrometers; where any lower limit can be paired with any upper limit of the range). Preferably, the particles are agglomerates of smaller particles. The particles preferably comprise crystallites that are less than about 1 micrometer in size (preferably, less than about 0.5 micrometer in size). The crystallites can be present as acicular crystallites, as raft-like structures comprising acicular crystallites, or as combinations of the acicular crystallites and raft-like structures. The concentration agents preferably have a surface area as measured by the BET (Brunauer-Emmett-Teller) method (calculation of the surface area of solids by physical adsorption of nitrogen gas molecules) that is greater than about 25 square meters per gram (m²/g), more preferably greater than about 50 m²/g. and most preferably greater than about 75 m²/g. The preferred agglomerated form of such particles can provide adsorptive capabilities of fine particle systems without the handling and other hazards often associated with fine particles. In addition, such agglomerate particles can settle readily in fluid and thus can provide rapid separation of microorganisms from a fluid phase (as well as allowing low back pressure if used in filtration applications).

In some embodiments, metal silicates can be used as concentration agents. Particularly useful metal silicates with a surface composition having a metal atom to silicon atom ratio of less than or equal to about 0.5, as determined by X-ray photoelectron spectroscopy (XPS). Preferably, the surface composition also comprises at least about 10 average atomic percent carbon, as determined by X-ray photoelectron spectroscopy (XPS). XPS is a technique that can determine the elemental composition of the outermost approximately 3 to 10 nanometers (nm) of a sample surface and that is sensitive to all elements in the periodic table except hydrogen and helium. XPS is a quantitative technique with detection limits for most elements in the 0.1 to 1 atomic percent concentration range. Preferred surface composition assessment conditions for XPS can include a take-off angle of 90 degrees measured with respect to the sample surface with a solid angle of acceptance of ±10 degrees.

When dispersed or suspended in water systems, metal silicates can exhibit surface charges that are characteristic of the material and the pH of the water system. The potential across the material-water interface is called the “zeta potential,” which can be calculated from electrophoretic mobilities (that is, from the rates at which the particles of material travel between charged electrodes placed in the water system). The concentration agents used in carrying out the method of the present disclosure have zeta potentials that are more negative than that of, for example, a common metal silicate such as ordinary talc. Yet the concentration agents are more effective than talc in concentrating microorganisms such as bacteria, the surfaces of which generally tend to be negatively charged. Preferably, the concentration agents have a negative zeta potential at a pH of about 7 (more preferably, a Smoluchowski zeta potential in the range of about −9 millivolts to about −25 millivolts at a pH of about 7; even more preferably, a Smoluchowski zeta potential in the range of about −10 millivolts to about −20 millivolts at a pH of about 7; most preferably, a Smoluchowski zeta potential in the range of about −11 millivolts to about −15 millivolts at a pH of about 7).

Examples of useful metal silicates include amorphous silicates of metals such as magnesium, calcium, zinc, aluminum, iron, titanium, and the like (preferably, magnesium, zinc, iron, and titanium; more preferably, magnesium), and combinations thereof. Preferred are amorphous metal silicates in at least partially fused particulate form (more preferably, amorphous, spheroidized metal silicates; most preferably, amorphous, spheroidized magnesium silicate). Metal silicates are known and can be chemically synthesized by known methods or obtained through the mining and processing of raw ores that are naturally-occurring.

Amorphous, at least partially fused particulate forms of metal silicates can be prepared by any of the known methods of melting or softening relatively small feed particles (for example, average particle sizes up to about 25 microns) under controlled conditions to make generally ellipsoidal or spheroidal particles (that is, particles having magnified two-dimensional images that are generally rounded and free of sharp corners or edges, including truly or substantially circular and elliptical shapes and any other rounded or curved shapes). Such methods include atomization, fire polishing, direct fusion, and the like. A preferred method is flame fusion, in which at least partially fused, substantially glassy particles are formed by direct fusion or fire polishing of solid feed particles (for example, as in the method described in U.S. Pat. No. 6,045,913 (Castle), the description of which is incorporated herein by reference). Most preferably, such methods can be utilized to produce amorphous, spheroidized metal silicates by converting a substantial portion of irregularly-shaped feed particles (for example, from about 15 to about 99 volume percent; preferably, from about 50 to about 99 volume percent; more preferably, from about 75 to about 99 volume percent; most preferably, from about 90 to about 99 volume percent) to generally ellipsoidal or spheroidal particles.

Some amorphous metal silicates are commercially available. For example, amorphous, spheroidized magnesium silicate is commercially available for use in cosmetic formulations (for example, as 3M Cosmetic Microspheres CM-111, available from 3M Company, St. Paul, Minn.).

In some embodiments, amorphous metal silicates can further comprise other materials including oxides of metals (for example, iron or titanium), crystalline metal silicates, other crystalline materials, and the like, provided that the concentration agents have the above-described surface compositions. The concentration agents, however, preferably contain essentially no crystalline silica.

In some embodiments, diatomaceous earth bearing, on at least a portion of its surface, a surface treatment comprising a surface modifier comprising titanium dioxide, fine-nanoscale gold or platinum, or a combination thereof for use as concentration agents.

Thus, concentration agents comprising certain types of surface-treated or surface-modified diatomaceous earth (namely, bearing a surface treatment comprising a surface modifier comprising titanium dioxide, fine-nanoscale gold or platinum, or a combination thereof) can be effective when compared to untreated diatomaceous earth for concentrating microorganisms. The surface treatment preferably further comprises a metal oxide selected from ferric oxide, zinc oxide, aluminum oxide, and the like, and combinations thereof (more preferably, ferric oxide). Although noble metals such as gold have been known to exhibit antimicrobial characteristics, the gold-containing concentration agents used in the process of the invention surprisingly can be effective not only in concentrating the microorganisms but also in leaving them viable for purposes of detection or assay.

Useful surface modifiers include fine-nanoscale gold; fine-nanoscale platinum; fine-nanoscale gold in combination with at least one metal oxide (preferably, titanium dioxide, ferric oxide, or a combination thereof); titanium dioxide; titanium dioxide in combination with at least one other (that is, other than titanium dioxide) metal oxide; and the like; and combinations thereof. Preferred surface modifiers include fine-nanoscale gold; fine-nanoscale platinum; fine-nanoscale gold in combination with at least ferric oxide or titanium dioxide; titanium dioxide; titanium dioxide in combination with at least ferric oxide; and combinations thereof.

More preferred surface modifiers include fine-nanoscale gold; fine-nanoscale platinum; fine-nanoscale gold in combination with ferric oxide or titanium dioxide; titanium dioxide; titanium dioxide in combination with ferric oxide; and combinations thereof (even more preferably, fine-nanoscale gold; fine-nanoscale gold in combination with ferric oxide or titanium dioxide; titanium dioxide in combination with ferric oxide; and combinations thereof). Fine-nanoscale gold, fine-nanoscale gold in combination with ferric oxide or titanium dioxide, and combinations thereof are most preferred. In some embodiments, useful concentration agents (e.g., dispersible particles) can have binding groups bound to such particles. The binding (e.g., functional) groups of the particles can have a specific affinity for specific microorganisms present in the samples. In some embodiments, the binding groups having more than site available for attaching multiple microorganisms present in a sample.

In some embodiments, the particles have magnetic properties. The particles can, for example, have magnetic cores. Microorganism bound compositions containing such concentration agents can be collected by the application of a magnetic field, for example, to transfer the composition from the first reservoir to the second reservoir of a sampling device.

In some embodiments, biomolecules (e.g., antibodies) can be covalently bonded to particles by any of a variety of methods for forming concentration agents. For example, glutaraldehyde, aldehyde-Schiff base, n-hydroxyl succinimide, azlactone, cyanogen bromide, maleic anhydride, etc., may be used as suitable attachment chemistries.

In some embodiments, biomolecules useful as concentration agents can be any chemical compound that naturally occurs in living organisms, as well as derivatives or fragments of such naturally occurring compounds. Biomolecules consist primarily of carbon and hydrogen, along with nitrogen, oxygen, phosphorus, and sulfur. Other elements sometimes are incorporated but are much less common. Biomolecules include, but are not limited to, proteins, antibodies, polypeptides, carbohydrates, polysaccharides, lipids, fatty acids, steroids, prostaglandins, prostacyclines, vitamins, cofactors, cytokines, and nucleic acids (including DNA, RNA, nucleosides, nucleotides, purines, and pyrimidines), metabolic products that are produced by living organisms including, for example, antibiotics and toxins. Biomolecules may also include derivatives of naturally occurring biomolecules, such as a protein or antibody that has been modified with chemicals (e.g., oxidized with sodium periodate). Biomolecules may also include crosslinked naturally occurring biomolecules, or a crosslinked product of a naturally occurring biomolecule with a chemical substance. Thus, as used herein, the term “biomolecule” includes, but is not limited to, both unmodified and modified molecules (e.g., glycosylated proteins, oxidized antibodies) and fragments thereof (e.g., protein fragments). Fragments of biomolecules can include those resulting from hydrolysis due to chemical, enzymatic, or irradiation treatments, for example.

In certain embodiments, biomolecules may be covalently bonded to one or more of the biomolecule-binding groups. In certain embodiments, the biomolecule includes or can be modified to include an aldehyde group prior to its attachment to the biomolecule-binding group.

The selective attachment of a target biological analyte can be achieved directly or it may be achieved through a capture agent, e.g., antigen-antibody binding (where the target biological analyte itself includes the antigen bound to an antibody immobilized on the detection surface).

Concentration agents having capture agents can include species (e.g., molecules, groups of molecules) that have high affinity for a target biological analyte, and preferably are specific for a target analyte. Capture agents include, for example, antibodies and fragments thereof (Fab, Fab′, Fc), polypeptides, aptamers, DNA, RNA, oligonucleotides, proteins, antibodies, carbohydrates, polysaccharides, lipids, fatty acids, steroids, vitamins, cytokines, lectins, cofactors, and receptors (e.g., phage receptors). Capture agents may also include derivatives of naturally occurring biomolecules, such as a protein or antibody that has been modified with chemicals. These may also include crosslinked naturally occurring biomolecules, or a crosslinked product of a naturally occurring biomolecule with a chemical substance.

Some biomolecule capture agents suitable for use in the present disclosure include polypeptides including antibodies, antibody conjugates, and proteins such as avidin, streptavidin, and clumping factor). In particular, biomolecule capture agents are antibodies. The term “antibody” is intended to include whole antibodies of any isotype (IgG, IgA, IgM, IgE, etc.), and fragments thereof from vertebrate, e.g., mammalian species, which are also specifically reactive with foreign compounds, e.g., proteins.

Bioaffinity pairs, such as antigen/hapten, antibody/antigen binding fragment of the antibody, or complementary nucleic acids, bioreceptor/ligand (interleukin-4 and its receptor) may be used to attach capture agents. One of the pairs of such biomolecules is covalently attached to the biomolecule-binding agent. These biomolecules form part of a “capture agent” for a target biological analyte. For example, the strong bond formed between biotin and avidin and/or streptavidin may be particularly useful when attaching an antibody to a surface. Preferably, streptavidin can be used as a means to attach an antibody, to a surface. Streptavidin is a tetrameric protein isolated from Streptomyces avidinii that binds tightly to the vitamin biotin. Proteins, such as streptavidin, can be attached to surfaces through a number of chemistries.

Concentration agents useful in the method of the present disclosure can be applied to a variety of different types of samples comprising microorganisms. Samples having low microorganism concentrations can have microorganisms within the sample concentrated as described herein. Some examples of samples can include, but not limited to, medical, environmental, food, feed, clinical, and laboratory samples, and combinations thereof. Medical or veterinary samples can include, for example, cells, tissues, or fluids from a biological source (for example, a human or an animal) that are to be assayed for clinical diagnosis. Environmental samples can be, for example, from a medical or veterinary facility, an industrial facility, soil, a water source, a food preparation area (food contact and non-contact areas), environmental surfaces (e.g., floors, walls, ceilings, fomites, equipment, water, water containers, and air filters), a laboratory, or an area that has been potentially subjected to bioterrorism. Food processing, handling, and preparation area samples, potable water and environmental surfaces are preferred, as these are often of particular concern in regard to food supply contamination by bacterial pathogens.

A sample useful in the method of the present disclosure can be in the form of a fluid (e.g., a liquid, or a dispersion or suspension). In some embodiments, samples obtained in the form of a liquid can be concentrated with a dispersible concentration agent so that microorganism bound composition can be formed. Preferred samples include foods, beverages, potable water, biological fluids, environmental samples, and combinations thereof (with foods, beverages, potable water, environmental samples, and combinations thereof being more preferred).

Additional descriptions of methods for concentrating microorganisms are described in International (PCT) Application No. U.S. 2009/069780, the disclosure of which is incorporated herein by reference for this purpose.

### Kits

In the current disclosure, kits are described that may include a holder device and a sampling device.

In some embodiments, a kit of the current disclosure may include a holder device 100, and a sampling device 500, and at least one thin film culture plate device 10.

In another embodiment, a kit of the current disclosure may include a holder device 100, and a sampling device 600, and at least one thin film culture plate device 10.

In another embodiment, a kit of the current disclosure may include a plurality of holder devices 100.

The above kits of the current disclosure may further include a concentrating agent. A suitable concentrating agent to include in a kit of the current disclosure may include any of: particles with affinity ligands; particles without affinity ligands; antibodies or antigen binding fragments; receptors; and combinations thereof. A suitable concentration agent for a kit of the current disclosure may include a dispersible concentration agent. In some embodiments, kits may include concentration agent may include any of gamma-FeO(OH), metal silicates, or diatomaceous earth. A concentration agent may include surface treated diatomaceous earth, the surface treatment selected from the group consisting of titanium dioxide, nanoscale gold, nanoscale platinum and combinations thereof.

Additional descriptions of sampling devices and concentrating agents useful for concentrating microorganisms, and useful for including in kits of the current disclosure, include those described in International (PCT) Application No. U.S. 2009/069780, the disclosure of which is incorporated herein by reference for this purpose.

In some embodiments, kits of the current disclosure may further include a detection agent and a growth medium.

### Methods of Using a Holder Device

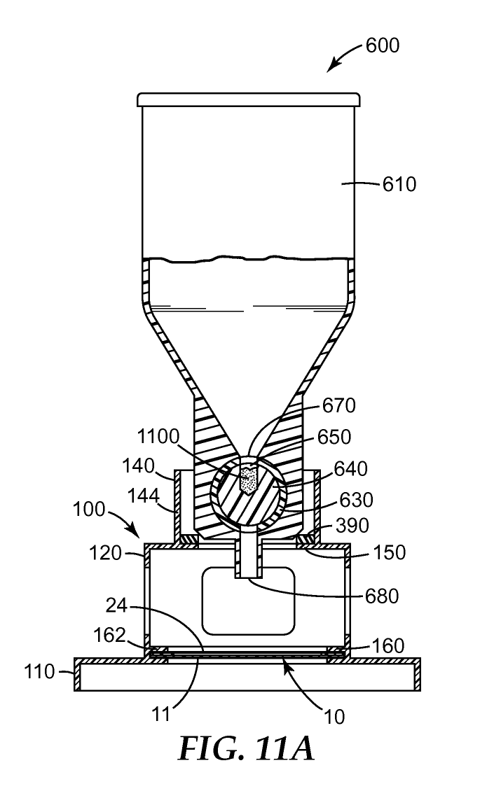

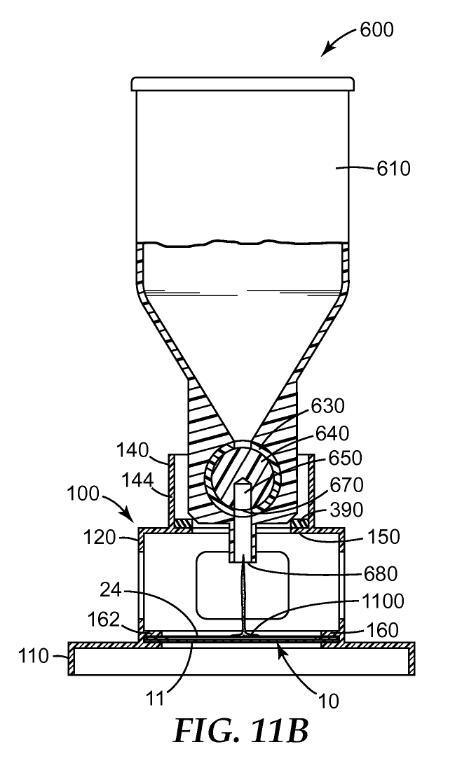

FIGS. 11A and 11B show an embodiment of using a combination of a holder device 100 (shown in partial cutaway view), a culture plate device 10 (shown in cutaway; cover sheet 22 is not in contact with sample zone 24), and a sampling device 600 to dispense a test sample 1100. In the embodiment shown in FIG. 11A, sampling device 600 is seated in upper housing portion 140 of holder device 100, resting on O-ring 390 on shoulder portion 150, and second external opening 680 is positioned over sample zone 24 of culture plate device 10. Second external opening 680 is shown as being located within lower housing portion 120. Sampling device 600 may optionally rest against a portion of circumferential wall 144, which may be dimensioned to aid in supporting sampling device 600 in an upright position. Test sample 1100, which may include a concentration agent of the current description, is shown in FIG. 11A as being settled from first reservoir 610 into second reservoir 650. Culture plate base member 11 is supported by first guide member portion 160 and second guide member portion 162, the entire combination being supported on base 110 of holder device 100.

FIG. 11B illustrates the dispensing of test sample 1100 from onto sample zone 24 of culture plate device 10, showing movable feature 640 within housing 630 rotated into a position having second opening 670 facing second external opening 680, and test sample 1100 falling from second external opening 680 onto sample zone 24.

The current disclosure includes methods for allowing a user to dispense an aqueous test sample from a sampling device to a thin film culture plate device. In an embodiment, the method includes steps of:

(a) providing a holder device 100 of the current disclosure;

(b) providing an aqueous test sample contained in a sampling device of the current disclosure, the sampling device having a second reservoir and a second external opening;

(c) providing a thin film culture plate device 10 of the current disclosure, including a plate base member 11 and a cover sheet 22 attached to plate base member 11 along an adhesive hinge 15;

(d) abutting a lower major surface of cover sheet 22 against first cover support member 180;

(e) inserting a front edge of plate base member 11 into horizontal slot 133 and urging plate base member 11 along an insertion pathway (see FIGS. 4A and 4B);

(f) inserting second external opening of the sampling device into upper housing portion 140; and

(g) dispensing the aqueous test sample from the second reservoir, through the second external opening and onto the plate base member.

In some embodiments of the above described method, cover sheet 22 is slightly longer than the plate base member 11, and abutting cover sheet 11 against first cover support member 180 is sufficient to initiate lifting cover sheet 180 from a closed position, without, for example, lifting the cover by hand.

In some embodiments of the above described method, step (e) includes visually confirming a positioning of thin film culture plate device 10 within lower housing portion interior region 120.

In some embodiments of the above described method, the method may include a step of withdrawing thin film culture plate device 10 from holder device 100, with a further embodiment of maintaining a substantially constant contact angle between cover sheet 22 and a top surface of the aqueous test sample located on plate base member 11 as the cover sheet returns to a closed position.

In another embodiment, as user may dispense a plurality of aqueous test samples from a sampling device to a corresponding plurality of thin film culture plate devices with minimal cross-contamination, by carrying out the above method, including the step of withdrawing thin film culture plate device 10 from holder device 100, and then repeating the dispensing method with another thin film culture plate device 10. The dispensing of a plurality of aqueous test samples from a sampling device to a corresponding plurality of thin film culture plate devices may be further facilitated by using an automation system for positioning of a culture plate device 10 and/or a sampling device of the current disclosure.

The current description includes the following items:

Item 1 is a holder device that allows a user to dispense a test sample from a sampling device to a culture plate, the culture plate including a culture plate base member having a sample zone and a cover sheet having a sample contact portion covering the sample zone, the holder device comprising:

(a) a housing comprising a support surface arranged to support the culture plate base member; and

(b) a first cover support member arranged in spaced relationship to the support surface, thereby to support a portion of the cover sheet, whereby at least a portion of the sample zone is exposed and a user is allowed to dispense a test sample into the sample zone.

Item 2 is the holder device of Item 1, wherein the support surface comprises first and second guide member portions that define a linear insertion path for the culture plate base member.

Item 3 is the holder device of Item 2, wherein the first and second guide member portions comprise first and second guide tracks, wherein each guide track has a first end and a second end, and the first ends of the guide tracks are arranged to define an insertion slot adjacent to a first end of the first cover support member.

Item 4 is the device of any one of Items 2 or 3, further comprising a stop member positioned to define an axial limit of the linear insertion path.

Item 5 is the holder device of any one of Items 2 to 4, further comprising a second cover support member in spaced relationship to the support surface, the first and second cover support members arranged to support side portions of the cover sheet.

Item 6 is the holder device of Item 5, wherein the first and second cover support members each comprise a ramp portion arranged to support a leading edge of the cover sheet when the culture plate base member is inserted into the insertion slot.

Item 7 is the holder device of any one of Items 5 or 6, wherein the housing comprises a front wall having an opening defined therein, and wherein side portions of the front wall comprise the first and second cover support members, whereby a gap is formed between the housing and the sample contact portion of the cover sheet.

Item 8 is the holder device of any one of Items 5 to 7, wherein the housing comprises a top wall having an opening defined therein to allow a user to dispense a test sample from a sampling device into an interior volume of the housing.

Item 9 is the holder device of any one of Items 5 to 8, wherein the housing comprises a lower housing portion, an upper housing portion defining a top opening, and a shoulder portion defining an opening between the lower portion and the upper portion, the shoulder portion and upper housing portion dimensioned to support a sampling device.

Item 10 is the holder device of Item 9, wherein the upper portion comprises a circumferential wall.

Item 11 is the holder device of Item 10 further comprising an O-ring seated on the shoulder portion and abutting an inner surface of the circumferential outer wall.

Item 12 is the holder device of Item 9 wherein the lower housing portion is substantially rectangular and further comprises a front wall, a back wall, first side wall, a second side wall, and a top wall that includes the shoulder portion.

Item 13 is the holder device of Item 12 wherein an observation opening is defined in any of the first side wall, the second side wall, and the back wall.

Item 14 is a holder device that allows a user to dispense a test sample from a sampling device to a culture plate, the culture plate including a culture plate base member having a sample zone and a cover sheet having a sample contact portion covering the sample zone, the holder device comprising:

(a) a housing, the housing comprising:
(i) a base portion having a support surface;
(ii) a lower housing portion extending upwards from the support surface, the lower housing portion defining a lower housing interior region and having a front wall, the front wall having an insertion slot defined therein;
(iii) an upper housing portion extending upwards from a top surface of the lower housing portion;
(iv) a shoulder portion between the upper housing portion and the lower housing portion, the shoulder portion defining a first opening;
(v) a guide member portion on an interior surface of the lower housing portion and dimensioned to support the culture plate base member, the guide member being aligned with the insertion slot and defining an insertion path; and

(b) a first cover support member arranged in spaced relation to the insertion slot; thereby to support a portion of the cover sheet, whereby at least a portion of the sample zone is exposed and a user is allowed to dispense a test sample into the sample zone.

Item 15 is the holder device of Item 14, further comprising a second cover support member in spaced relationship to the support surface, the first and second cover support members arranged to support side portions of the cover sheet.

Item 16 is a testing kit comprising:
(a) the holder device of any one of Items 1 to 15;
(b) at least one thin film culture plate device.

Item 17 is the testing kit of Item 16, further comprising a sampling device, wherein the sampling device comprises:

a first reservoir having a first opening and at least one resealable external opening;

a second reservoir having a second opening;

an element comprising
i) a housing; and
ii) a movable feature residing within the housing, the movable feature having at least a first location and a second location,

wherein an interior of the second reservoir is located within the movable feature and the second opening resides on the exterior of a portion of the movable feature, the first reservoir located above the element when the sampling device is in an upright position; and

a second external opening, the second external opening located below the element when the sampling device is in the upright position, wherein at the first location a first passageway connects the first reservoir to the second reservoir so that the first opening is in fluid communication with the second opening, wherein at the second location a second passageway connects the second reservoir to the second external opening so that the second opening is in fluid communication with the second external opening.

Item 18 is the testing kit of Item 16, further comprising a sampling device, wherein the sampling device comprises:

a first reservoir having a first opening, a first resealable external opening and a first volume;

a second reservoir having a second opening, a second external opening and a second volume; and

a plunger having a seal, the seal residing on a portion of the plunger proximate to a distal end of the plunger, the seal isolating the second volume of the second reservoir from the first volume of the first reservoir, the second volume removed through the second external opening, wherein the first reservoir is located above the second reservoir when the sampling device is in an upright position.

Item 19 is the testing kit of any one of Items 17 or 18, wherein a ratio of the first volume to the second volume is in a range of about 10:1 to about 1000:1.

Item 20 is the testing kit of Item 19, wherein the second volume is about 1 milliliter.

Item 21 is the testing kit of any one of Items 16 to 19, further comprising a concentration agent wherein the concentration agent is a dispersable particulate material selected from the group consisting of particles with affinity ligands, particles without affinity ligands, antibodies or antigen binding fragments, receptors and combinations thereof.

Item 22 is the testing kit of Item 21, wherein the concentration agent comprises metal silicates.

Item 23 is the testing kit of Item 21, wherein the concentration agent comprises spheroidized magnesium silicate.

Item 24 is the testing kit of Item 21, wherein the concentration agent comprises surface treated diatomaceous earth, the surface treatment selected from the group consisting of titanium dioxide, nanoscale gold, nanoscale platinum and combinations thereof.

Item 25 is the testing kit of Item 21, wherein the concentration agent comprises gamma-FeO(OH).

Item 26 is the testing kit of any one of Items 16 to 25, further comprising a detection agent and a growth medium.

Item 27 is the testing kit of any one of Items 16 to 26 further comprising a plurality of holder devices.

Item 28 is a method that allows a user to dispense an aqueous test sample from a sampling device to a thin film culture plate device, the method comprising steps of:

(a) providing a holder device of any one of Items 8 to 15;

(b) providing a test sample contained in a sampling device having a second reservoir and a second external opening;

(c) providing a culture plate device including a plate base member having a sample zone and a cover sheet having a sample contact portion covering the sample zone

(d) abutting a portion of the cover sheet against the first cover support member;

(e) inserting a leading edge of the plate base member into the insertion slot and urging the plate base member along the insertion path;

(f) inserting the second external opening of the sampling device into an opening in the housing; and

(g) dispensing the aqueous test sample from the second reservoir onto the plate base member.

Item 29 is the method of Item 28 wherein the cover sheet has a tab portion extending beyond the leading edge of the plate base member, and abutting the tab portion against the first cover support member thereby to lift a portion of the cover sheet from the culture plate base member.

Item 30 is the method of any one of Items 28 or 29 wherein step (e) further comprises visually confirming a positioning of the thin film culture plate device within the lower housing portion.

Item 31 is the method of any one of Items 28 to 30, further comprising after step (g) a step of sliding the culture plate device back along the insertion path and out of the holder device.

Item 32 is the method of Item 31, further comprising repeating the method of Item 25 with another test sample and another culture plate device.

Item 33 is the method of Item 32, further comprising using an automation system to position the culture plate device and/or the sampling device.

### EXAMPLES

### Example 1

### Holder Device

A model for holder device 100 was constructed using Pro/Engineer Wildfire 4 solids modeling software supplied by Parametric Technologies Corp. (Needham, Mass.). When the model was completed and checked the model file was converted to an .stl file. STL is a file format native to the stereolithography CAD software created by 3D Systems (Rock Hill, S.C.). This file format supported by many other software packages is widely used for rapid prototyping and computer-aided manufacturing. STL files describe only the surface geometry of a three dimensional object without any representation of color, texture or other common CAD model attributes. The holder device part was built using a 3D Systems Model 250 SLA machine (Rock Hill, S.C.). The resin used in the machine was DSM Somos 9420 (an epoxy resin, obtained from DMS Somos Elgin, Ill.). After removal from the machine the part was cleaned and sanded to remove imperfections.

### Example 2

### Testing for Reduction in Inoculation Variability Using the Holder Device

A set of 3M™ Petrifilm™ E. coli/Coliform Count Plates (n=5, obtained from 3M Company, St. Paul, Minn.) were each inoculated with 1 ml tap water, without using the holder device, and the cover sheets were each dropped manually to close the culture plate.

Another set of five 3M™ Petrifilm™ E. coli/Coliform Count Plates (3M Company, St. Paul, Minn.) were mounted one at a time in the holder device of Example 1, resulting in the top cover being held away from the inoculation area. Each plate was inoculated with 1 ml tap water, and removed from the stand gradually so as to drop the cover sheet to close the plate.

After 10 minutes of incubation at room temperature (about 23° C.) to allow gel rehydration, all plates were analyzed by using a 3M™ Petrifilm™ Plate Reader (automated detection) per manufacturers instructions. Images from the automated reader were analyzed using IMAGEPRO software (Media Cybernetics, Bethesda, Md.) wherein each image was evaluated for the number of bubbles, detected as white objects against a red background in the image. The size of each bubble in square millimeters (mm²) was determined by the software after calibrating the image to known size scale. Results, reported in number of bubbles and average size (in square millimeters), are listed in Table 1 below.

TABLE 1
Reduction in air bubble entrapment during
inoculation by using a Holder Device

| Inoculation | | Bubble | Average Size |
|---|---|---|---|
| Method | Replicate | Number | (mm²) |
| Manual | Inoculation 1 | 4 | 0.8 |
| Manual | Inoculation 2 | 14 | 1.5 |
| Manual | Inoculation 3 | 4 | 0.7 |
| Manual | Inoculation 4 | 6 | 2 |
| Manual | Inoculation 5 | 19 | 0.6 |
| Holder device | Inoculation 1 | 0 | 0 |
| Holder device | Inoculation 2 | 0 | 0 |
| Holder device | Inoculation 3 | 2 | 0.2 |
| Holder device | Inoculation 4 | 4 | 0.2 |
| Holder device | Inoculation 5 | 0 | 0 |

Although the present disclosure is herein described in terms of specific embodiments, it will be readily apparent to those skilled in the art that various modifications, rearrangements, and substitutions can be made without departing from the spirit of the invention. The scope of the present invention is thus only limited by the claims appended herein.

What is claimed is:

1. A holder device that allows a user to dispense a test sample from a sampling device to a culture plate, the culture plate including a culture plate base member having a sample zone and a cover sheet having a sample contact portion covering the sample zone, the holder device comprising:
(a) a housing, the housing comprising:
(i) a base portion having a support surface;
(ii) a lower housing portion extending upwards from the support surface, the lower housing portion defining a lower housing interior region and having a front wall, the front wall having an insertion slot defined therein;
(iii) an upper housing portion extending upwards from a top surface of the lower housing portion;
(iv) a shoulder portion between the upper housing portion and the lower housing portion, the shoulder portion defining a first opening;
(v) a guide member portion on an interior surface of the lower housing portion and dimensioned to support the culture plate base member, the guide member being aligned with the insertion slot and defining an insertion path; and
(b) a first cover support member arranged in spaced relation to the insertion slot; thereby to support a portion of the cover sheet, whereby at least a portion of the sample zone is exposed and a user is allowed to dispense a test sample into the sample zone.

2. The holder device of claim 1, further comprising a second cover support member in spaced relationship to the support surface, the first and second cover support members arranged to support side portions of the cover sheet.

3. A testing kit comprising:
(a) the holder device of claim 1;
(b) at least one thin film culture plate device.

4. The testing kit of claim 3, further comprising a sampling device, wherein the sampling device comprises:
a first reservoir having a first opening and at least one resealable external opening;
a second reservoir having a second opening;
an element comprising
i) a housing; and
ii) a movable feature residing within the housing, the movable feature having at least a first location and a second location,
wherein an interior of the second reservoir is located within the movable feature and the second opening resides on the exterior of a portion of the movable feature, the first reservoir located above the element when the sampling device is in an upright position; and
a second external opening, the second external opening located below the element when the sampling device is in the upright position, wherein at the first location a first passageway connects the first reservoir to the second reservoir so that the first opening is in fluid communication with the second opening, wherein at the second location a second passageway connects the second reservoir to the second external opening so that the second opening is in fluid communication with the second external opening.

5. The testing kit of claim 3, further comprising a sampling device, wherein the sampling device comprises:
a first reservoir having a first opening, a first resealable external opening and a first volume;
a second reservoir having a second opening, a second external opening and a second volume; and
a plunger having a seal, the seal residing on a portion of the plunger proximate to a distal end of the plunger, the seal isolating the second volume of the second reservoir from the first volume of the first reservoir, the second volume removed through the second external opening, wherein the first reservoir is located above the second reservoir when the sampling device is in an upright position.

6. The testing kit of claim 5, wherein a ratio of the first volume to the second volume is in a range of about 10:1 to about 1000:1.

7. The testing kit of claim 6, wherein the second volume is about 1 milliliter.

8. The testing kit of claim 4, further comprising a concentration agent wherein the concentration agent is a dispersable particulate material selected from the group consisting of particles with affinity ligands, particles without affinity ligands, antibodies or antigen binding fragments, receptors and combinations thereof.

9. The testing kit of claim 8, wherein the concentration agent comprises metal silicates.

10. The testing kit of claim 8, wherein the concentration agent comprises spheroidized magnesium silicate.

11. The testing kit of claim 8, wherein the concentration agent comprises surface treated diatomaceous earth, the surface treatment selected from the group consisting of titanium dioxide, nanoscale gold, nanoscale platinum and combinations thereof.

12. The testing kit of claim 8, wherein the concentration agent comprises gamma-FeO(OH).

13. The testing kit of claim 4, further comprising a detection agent and a growth medium.

14. The testing kit of claim 4, further comprising a plurality of holder devices.

15. A method that allows a user to dispense an aqueous test sample from a sampling device to a thin film culture plate device, the method comprising steps of:
(a) providing a holder device of claim 1;
(b) providing a test sample contained in a sampling device having a second reservoir and a second external opening;
(c) providing a culture plate device including a plate base member having a sample zone and a cover sheet having a sample contact portion covering the sample zone
(d) abutting a portion of the cover sheet against the first cover support member;
(e) inserting a leading edge of the plate base member into the insertion slot and urging the plate base member along the insertion path;
(f) inserting the second external opening of the sampling device into an opening in the housing; and
(g) dispensing the aqueous test sample from the second reservoir onto the plate base member.

16. The method of claim 15 wherein the cover sheet has a tab portion extending beyond the leading edge of the plate base member, and abutting the tab portion against the first cover support member thereby to lift a portion of the cover sheet from the culture plate base member.

17. The method of claim 15 wherein step (e) further comprises visually confirming a positioning of the thin film culture plate device within the lower housing portion.

18. The method of claim 15, further comprising after step (g) a step of sliding the culture plate device back along the insertion path and out of the holder device.

19. The method of claim 18, further comprising repeating the method of claim 18 with another test sample and another culture plate device.

20. The method of claim 19, further comprising using an automation system to position the culture plate device and/or the sampling device.
